(19) United States
(12) Patent Application Publication
(10) Pub. No.: US 2009/0054722 A1
(43) Pub. Date: Feb. 26, 2009

(54) Drug Carrier Containing Magnetic Fine Particles and System Using the Same

(76) Inventors: Ryoko Sugano, Kodaira (JP); Nami Sugita, Ranzan (JP); Teruo Kohashi, Hachioji (JP); Keiji Takata, Suita (JP); Chiharu Mitsumata, Takasaki (JP); Shigeo Fujii, Kumagaya (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/186,545

(22) Filed: Aug. 6, 2008

(30) Foreign Application Priority Data
Aug. 24, 2007 (JP) 2007-218576

(51) Int. Cl. A61N 2/00; A61K 9/14; A61K 9/127

(52) U.S. Cl. 600/12; 424/490; 424/497; 424/450

(57) ABSTRACT

The present invention provides drug carriers having high heating efficiency by high-frequency dielectric heating in a state of being selectively accumulated in a target site. The drug carriers each consist of a drug, magnetic fine particles, and a shell containing the drug and the magnetic fine particles. The shell has an outer diameter in a range from 10 nm to 200 nm. The magnetic fine particles having an average particle diameter of d has a standard deviation σ of particle diameter distribution satisfying 0.8d>σ>0.4d. The magnetic fine particles contained in the individual drug carriers generate hysteresis heat due to high-frequency dielectric heating by irradiation of a high-frequency magnetic field.

### CLAIM OF PRIORITY

The present application claims priority from Japanese patent application JP 2007-218576 filed on Aug. 24, 2007, the content of which is hereby incorporated by reference into this application.

### BACKGROUND OF THE INVENTION

1. Field of the Invention

The present invention relates to a drug carrier containing magnetic fine particles which aims to improve drug release efficiency in a drug delivery system (hereinafter, referred to as DDS) and heat-generating efficiency in hyperthermia therapy by using site-oriented high-frequency dielectric heating in a field of medical technology, and relates to therapy equipment using the drug carrier.

2. Description of the Related Art

In DDS, drug targeting can be achieved by selectively delivering a drug only to a specific cell, tissue, or organ by use of carriers. In drug targeting, while the concentration of a drug in a treatment site is increased so that target pharmacological actions can be enhanced, an amount of the drug delivered to other sites is reduced so that side effects can be reduced. In addition to drug targeting, it is also required to preferably control a drug release rate and the like at a target tissue or organ by use of external stimulation in order to attain locally-specific effective drug efficacy. Especially, as drug carriers which are capable of increasing its accumulation selectively to a target site in response to temperature and further capable of controlling drug release, thermoresponsive materials (Japanese Patent Application Publication No. Hei. 9-169850), such as a thermosensitive polymeric micelle, and a thermoresponsive liposome (Japanese Patent Application Publication No. 2003-212755) have been investigated. Under present circumstances, these drug carriers are considered to be effective for accumulation of drug carriers at, and sustained release of drug carriers to, an affected area having a temperature different from that in a normal area.

In the meantime, a high-frequency dielectric heating method in hyperthermia (hyperthermia therapy for cancer) taking advantage of the nature that cancer cells are more susceptible to heat than normal cells is a method in which a living body is sandwiched by electrodes, and the entire living body is heated to approximately 42° C. The advantage of this treatment method is to be less invasive than surgical procedures and have lower impact on a patient. However, the cooling effect of hepatic perfusion does not allow a rise in the temperature inside a tumor; therefore, the tumor cannot be successfully coagulated and necrotized. In addition, since not only a tumor but also the entire living body is heated, there arises a problem of impact on normal cells in the case of continuous and long-term treatment. Against such a background, a high-frequency dielectric heating method has been examined (Japanese Patent Application Publication No. 2006-116083). In the method which takes advantage of heat-generating effect due to magnetic hysteresis loss of a ferromagnetic body in an alternating-current magnetic field, a magnetic powder incorporated into a tumor is heated to 60 to 80° C. so that only the tumor can be selectively coagulated and necrotized. Achieving such a result, this method is predicated on introducing a magnetic body serving as a body to be heated into a lesion site. However, in the case of using a magnetic powder having a size in a range from 1 μm to 1 mm, which is expected to demonstrate a high heat-generating effect due to huge hysteresis loss, it is necessary to introduce a heat-generating body directly into an affected area by an open surgery or a catheter (Japanese Patent Application Publication No. 2005-160749). This method has a great impact on a patient, and is not applicable to a lesion site situated in a deep part where an operation cannot be performed and a catheter cannot reach. Under these circumstances, in order to incorporate a magnetic body into a target site by a minimally invasive DDS, researches have been recently made on a drug containing magnetic fine particles based on nano-size magnetic fine particles serving as a magnetic body, in complex with a material adaptable to a living body, such as phospholipids, proteins, and water-soluble polymers (Japanese Patent Application Publication No. Hei. 3-128331).

In addition, it is necessary to monitor a heating condition for appropriate local heating, of a target site, by irradiation of a high-frequency magnetic field using magnetic fine particles as a body to be heated. As for monitoring the temperature in a living body, a method for measuring a temperature using a nuclear magnetic resonance imaging (hereinafter referred to as MRI) apparatus is disclosed in Japanese Patent Application Publication No. 2000-300535, for example.

### SUMMARY OF THE INVENTION

As for a method using a drug carrier having a thermoresponsive function, since thermosensitive phase transition in a living body takes a long time, it has not been achieved that a drug release rate is preferably controlled by heating a target site locally during treatment after drug carriers are selectively accumulated to the target site.

Meanwhile, as for a method using heat-generating effect due to magnetic hysteresis loss of magnetic fine particles, since the heat-generating efficiency due to hysteresis is lowered as the size of the magnetic fine particles is reduced. Accordingly, the method has not attained effective therapeutic efficacy yet. At the present time, no minimally-invasive heating technique having effective hyperthermia effect limited to a local site has been established; thus, a highly-efficient technique for heating a local site is demanded. Especially, it is effective to select a magnetic body having a high magnetic heating efficiency in order to improve a heating efficiency of a local site. However, although utilizing magnetic fine particles, a conventional drug containing magnetic fine particles is mainly based on a modified function added to the magnetic fine particles not on the magnetization characteristics of the magnetic fine particles. Accordingly, the magnetic fine particles constituting the drug carrier have not been sufficiently examined in terms of particle diameter distribution, magnetic heating efficiency, and the like which determine powder characteristics.

In order to rapidly attain therapeutic effect of hyperthermia and locally-specific effective drug efficacy of a thermoresponsive drug carrier while minimizing impact on a patient, it is essentially required to preferably control heating of a local site. As one of the measures to fulfill the requirement, it is effective that magnetic fine particles contained in a drug carrier have a high magnetic heating efficiency.

However, since a heating material is selected from preexisting materials which are available or modifiable, the heating material and its heat-generating characteristics vary according to the shape and the size. Furthermore, magnetization characteristics of a single particle and magnetization characteristics in a condensed system in which multiple particles aggregate also vary. Therefore, it is necessary to check whether or not a selected material can be used by performing characteristics analysis.

The present invention has been conducted in view of the above-described problems. A technical object of the present invention is to provide a drug carrier having a high magnetic heating efficiency in a state where the drug carriers are accumulated selectively to a target site, and to provide therapy equipment capable of heating a local site by use of the drug carriers in accordance with a high-frequency dielectric heating method.

On the basis of the above-described object, the present inventor focused on aggregation property and particle diameter distribution of an assembly of single magnetic-domain magnetic fine particles of nanometer order, and investigated particle diameter distribution and aggregation condition at which coercivity as shown in a hysteresis curve is enhanced. To be more specific, regarding an assembly state of single magnetic-domain magnetic fine particles having an average distance between particles of 323 nm and an average particle diameter of 75 nm, a magnetization curve was calculated with a standard deviation of the particle diameter distribution as a parameter on the basis of a model which incorporated an anisotropic energy, an applied magnetic-field energy, and an interparticle magnetic dipolar interaction energy of the whole system. Hysteresis loss was estimated according to the area of a hysteresis loop of the magnetization curve. As a result, the relationship between particle diameter distribution and hysteresis loss, as shown in FIG. 3, of magnetic fine particles in a condensed system was obtained. As the standard deviation of the particle diameter distribution is increased, hysteresis loss is increased. Moreover, when the standard deviation of the particle diameter distribution exceeds 0.4-times of the average particle diameter, the rate of increase is rapid. According to this result, it is possible to provide a drug carrier achieving a high magnetic heating efficiency by giving nonuniformity to the particle diameter distribution of an assembly of magnetic fine particles contained in the drug carrier, and to provide therapy equipment having a high heating efficiency which uses the drug carrier and a high-frequency dielectric heating method.

To be more specific, a drug carrier of the present invention includes: a drug, multiple magnetic fine particles which are aggregated; and a shell containing the drug and the multiple magnetic fine particles. The magnetic fine particles are single magnetic-domain magnetic fine particles, and the standard deviation σ of the magnetic fine particles satisfies 0.8d>σ>0.4d when d is the average particle diameter. The shell has an outer diameter in a range from 10 nm to 200 nm. The magnetic fine particles contained in the drug carriers generate hysteresis heat due to high-frequency dielectric heating by irradiation of a high-frequency magnetic field.

Meanwhile, therapy equipment of the present invention includes: a holding table for holding a test body to which the drug carriers have been administered; a high-frequency magnetic field irradiation unit for applying high-frequency dielectric heating to the drug carriers aggregated at a target site of the test body; a temperature monitor for monitoring the temperature of the target site; a control unit for causing the high-frequency magnetic field irradiation unit to operate until a rise in the temperature monitored by the temperature monitor reaches a predetermined target value of rise in temperature and for bringing the high-frequency magnetic field irradiation unit to a halt when the temperature rise reaches the target value of rise in temperature.

By giving nonuniformity of 0.8d>σ>0.4d to the particle diameter distribution of an assembly of the magnetic fine particles, it is possible to apply high-efficiency local heating to the drug carriers, which remain in blood vessels, at the target lesion site and to promote drug release specifically to the target site. Moreover, it is possible to shorten an exposure time in hyperthermia therapy for cancer and the like; thus, impact on a patient can be reduced.

A drug carrier containing magnetic fine particles according to the present invention demonstrates magnetic characteristics of high magnetic heating efficiency, and thereby enables heating by a short-term exposure or heating at a lower magnetic-field intensity. Accordingly, impact on a surrounding part adjacent to the target part can be reduced, and, as a result, minimally-invasive treatment can be performed. In addition, it is possible to provide treatment to an affected area to which a surgery cannot be performed. Moreover, providing treatment with a low magnetic field in a short period of time, the equipment can be operated at low power consumption.

### BRIEF DESCRIPTION OF THE DRAWINGS

FIG. 1 is a drawing showing magnetization curves of a magnetic fine particle condensed system for various volume fractions.

FIG. 2 is a drawing showing magnetization curves of a magnetic fine particle condensed system with particle diameter distributions as a parameter.

FIG. 3 is a drawing showing standard deviation dependency of hysteresis loss in a magnetic fine particle condensed system.

FIG. 4 is a schematic illustration of drug release from a drug carrier having a shell consisting of a thermoresponsive polymer.

FIG. 5 is a flowchart of an example of therapy method using drug carriers of the present invention.

FIG. 6 is an explanatory drawing of an example configuration of a static magnetic field gradient generation part in heat therapy equipment.

FIG. 7 is a drawing illustrating an example configuration of an alternating-current magnetic field generation part for irradiating a target site with a high-frequency magnetic field in the therapy equipment.

FIG. 8 is a schematic illustration of an example configuration of heat therapy equipment using drug carriers of the present invention.

FIGS. 9A and 9B are schematic illustrations of drug release from drug carriers delivered to the vicinity of a target site through blood vessels, when high-frequency magnetic field irradiation is applied to the drug carriers.

FIGS. 10A and 10B are schematic illustrations of drug release from drug carriers which have been delivered through blood vessels, penetrated through vessel walls in the vicinity of a target site, and accumulated in tissues at a target site, when high-frequency magnetic field irradiation is applied to the drug carriers.

FIGS. 11A and 11B are schematic illustrations of drug release from drug carriers, which have been delivered through blood vessels and accumulated in the vicinity of a target site, invading in a cell when high-frequency magnetic field irradiation is applied to the drug carriers.

### DESCRIPTION OF THE PREFERRED EMBODIMENTS

More detailed description will be given of a configuration of the present invention as follows.

In the present invention, the size of an effective drug carrier containing magnetic fine particles is in a range from 5 nm to 20 nm. If the size falls below 5 nm, the carrier is discharged by renal filtration. If the size is above 200 nm, the carrier is discharged by detoxifying process in the liver. The size is preferably in a range from 10 nm to 200 nm. In the case of aiming only to locally-specific drug release by DDS, it is not necessary to heat surrounding tissues, and a rise in temperature is limited to the drug carrier. Accordingly, the heating value required for the treatment is reduced compared to hyperthermia. On the other hand, in the case where the size of the drug carrier is 5 nm or more, the blood vessel permeability is higher when the size is smaller. The size of drug carrier specifically used for locally-specific release for the purpose of increasing the concentration of the drug is preferably in a range from 10 nm to 50 nm.

Magnetic fine particles constituting the drug carrier containing magnetic fine particles of the present invention preferably have an anisotropic magnetic field Hₖ with a small dispersion. Preferred dispersion range is in 0.01 or below.

An enhancement effect on hysteresis loss caused by high-frequency dielectric heating of a drug carrier containing magnetic fine particles of the present invention is achieved with the use of nonuniformity in particle diameters of the magnetic fine particles in a condensed system. This effect is exerted, as a result of competition between interparticle interaction and anisotropic energy of a single magnetic fine particle, under a condensed system having a high volume fraction in which the interaction is dominant. Preferably, the effect is utilized in the state where the volume fraction Φ satisfies the following relationship. To be more specific, the relationship is represented by the following formula (1). If this state is not satisfied, no significant enhancement effect on hysteresis loss can be expected.

$$φ > \frac{3 {H}_{k}}{{M}_{s} {μ}_{0}} \qquad ( 1 )$$

Here, the volume fraction is a product of the ratio of the volume V_{carrier} of a drug carrier containing magnetic fine particles to the volume V_{cluster} of an aggregate of the drug carriers containing magnetic fine particles in a condensed system at a target site multiplied by the number N_{carrier} of the drug carriers containing magnetic fine particles forming the aggregate, or a product of the ratio of the average volume V_{particle} of magnetic fine particles constituting a drug carrier containing magnetic fine particles to the volume V_{carrier} of the drug carrier containing magnetic fine particles multiplied by the number N_{particle} of the magnetic fine particles. To be more specific, the volume fraction is expressed by either the following formulas (2) or (3):

$$\begin{matrix}φ = {N}_{carrier} \frac{{V}_{carrier}}{{V}_{cluster}} & ( 2 ) \\ φ = {N}_{particle} \frac{{V}_{particle}}{{V}_{carrier}} & ( 3 )\end{matrix}$$

Magnetic fine particles constituting a drug carrier containing magnetic fine particles of the present invention preferably have a large ratio Mₛ/Hₖ between the saturated magnetization Mₛ and the anisotropic magnetic field Hₖ. Preferred is pure iron having a high saturated magnetization.

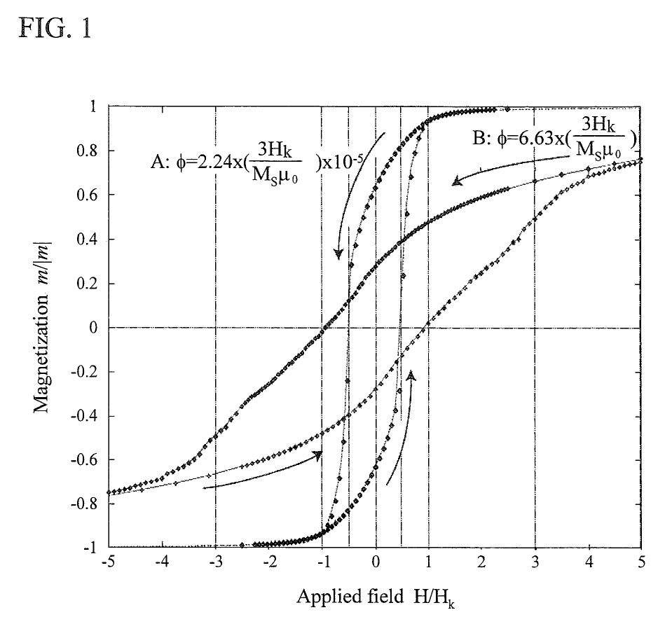

FIG. 1 shows a typical example of changes in magnetization curves according to the various volume fractions in the case of uniform particle diameter. Compared to the case of A where the volume fraction is so small that the effect by interparticle interaction can be mostly ignored, the coercivity in the case of B where the volume fraction is high increases as the area of the hysteresis loop is enlarged, reaching approximately double of that in the case where the effect of interparticle interaction can be ignored. The enhancement effect on hysteresis loss caused by high-frequency dielectric heating is exerted in a region where the effect of such interparticle interaction is significant. This is because two neighboring fine particles turn around in a pair by magnetic dipolar interaction. Accordingly, a reverse magnetic field Hᵣᵥ increases as the interparticle interaction is enhanced, and the coercivity becomes below the reverse magnetic field. In addition, the interparticle interaction is limited to the case where the average interparticle distance in a condensed system is approximately equal to the particle diameter. Accordingly, the following relationship regarding the coercivity H_{c} involving the maximum reverse magnetic field Hᵣᵥ^{max} is true:

$$\frac{{H}_{c}}{{H}_{k}} < {H}_{rv}^{max} = \frac{{M}_{s} {μ}_{0}}{12 {H}_{k}} \qquad ( 4 )$$

In the case of iron fine particles, the coercivity H_{c} is approximately 5 times the anisotropic magnetic field Hₖ. Meanwhile, in the case where there is no interparticle interaction bringing in the enhancement effect of hysteresis loss, there is no correlation among easily-magnetizable axes of individual fine particles in an aggregated powder compacting state during the production of the fine particles. Since the directions of the easily-magnetizable axes are random, the coercivity H_{c} in this case is approximately half the anisotropic magnetic field Hₖ. In other words, in a uniform powder compacting state of the fine particles, the coercivity H_{c} is increased to approximately equal to the anisotropic magnetic field Hₖ due to the effect of interparticle interaction.

Therefore, magnetic fine iron particles according to the present invention include those having a coercivity H_{c} in an aggregated powder compacting state during the production of the fine particles is in a range from approximately equal to the anisotropic magnetic field Hₖ to 5 times the anisotropic magnetic field Hₖ. Preferably, the magnetic fine iron particles include those having a coercivity H_{c} in an aggregated powder compacting state during the production of the fine particles of approximately double the coercivity of an aggregate at a ultra-low density.

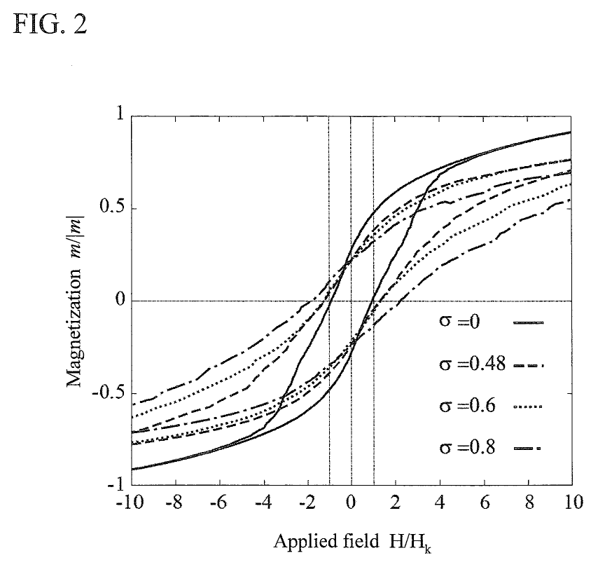

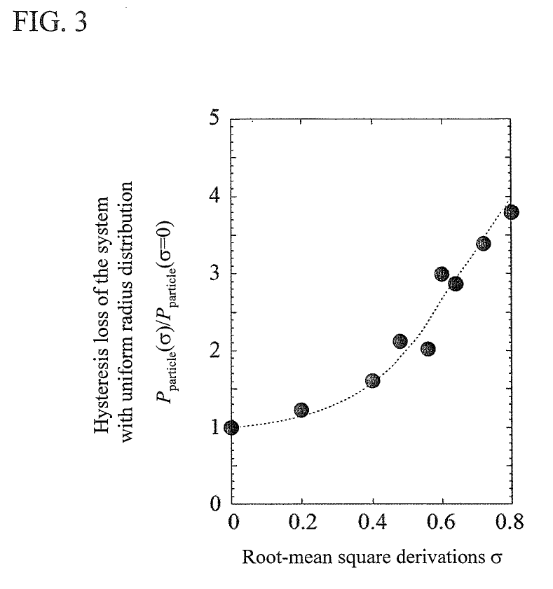

Meanwhile, the nonuniformity in particle diameters of drug carriers containing magnetic fine particles of the present invention is represented by nonuniformity in the saturated magnetization distribution. A particle having a large particle diameter has a strong effect of interaction in a larger range compared to the case where the particle diameters are uniform, and further has a high saturated magnetization. Accordingly, such a particle is highly resistant to a reverse magnetic field, and promotes an enhancement of the coercivity. As a result, an enhancement of coercivity and an enlargement of a hysteresis loop region are caused due to an increase in nonuniformity as shown in FIG. 2. Consequently, the relationship between the particle diameter distribution and hysteresis loss as shown in FIG. 3 can be obtained.

In the case of uniform particle diameter, the heating value Wh_{particle} per particle unit regarding magnetic fine particles constituting a drug carrier containing magnetic fine particles is expressed by the following formula (5) using a frequency f and hysteresis loss P_{particle} during dielectric heating, and the heating value Wh_{particle} per one drug carrier containing magnetic fine particles is expressed by the following formula (6):

Wh_{particle}(σ=0)=fP_{particle}>fHₖ²×10⁷ [Wm⁻³] (5)

Wh_{carrier}(σ=0)=Wh_{particle}(σ=0)>fφHₖ²×10⁷ [Wm⁻³] (6)

In the meantime, in the case where the particle diameter is nonuniform, hysteresis loss P_{particle} changes as shown in FIG. 3, and increases to 1.6-times of that in the case of uniform diameter at σ≈0.4, and to 4-times that in the case of uniform diameter at σ≈0.8. Moreover, the increase rate changes around σ≈0.4, and the increase rate of the line shape is approximately 1.5 when σ is 0.4 or below, while the linear increase rate increases to 5.5 when σ is 0.4 or above.

Magnetic fine particles constituting a drug carrier containing magnetic fine particles of the present invention preferably has an average diameter d in a range from 10 nm to 50 nm, and a standard deviation in a range from 0.4d to 1.0d. More preferably, the average particle diameter is in a range from 10 nm to 20 nm and the standard deviation is 0.4d or above. Further preferably, the average particle diameter is 10 nm and the standard deviation is 8 nm.

When the average particle diameter of an assembly of magnetic fine particles contained in the individual carriers i is defined as dᵢ, a preferred embodiment of the present invention includes a drug carrier containing magnetic fine particles which have a standard deviation σᵢ of particle diameters in the individual carriers i satisfying 0.8dᵢ>σᵢ>0.4dᵢ.

Furthermore, in the preferred embodiment of the present invention, in the case of aiming only for locally-specific drug release, magnetic fine particles contained in a drug carrier preferably have an average particle diameter of 5 nm and a standard deviation of 4 nm.

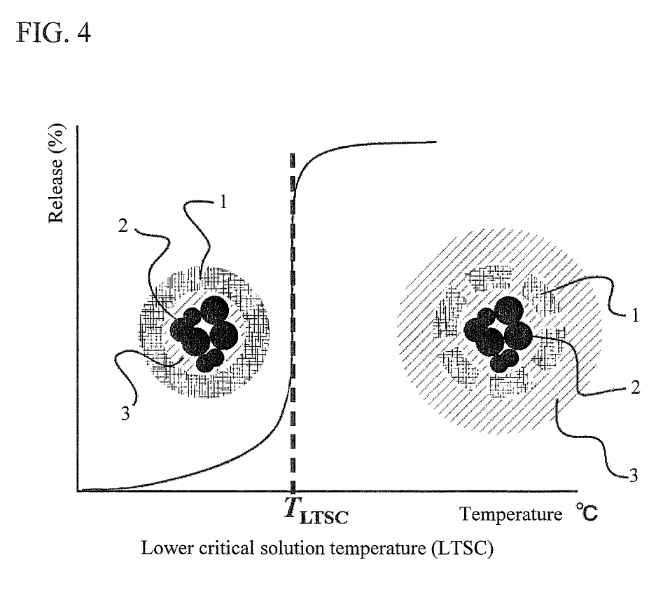

In the preferred embodiment of the present invention, a shell of the drug carrier containing magnetic fine particles consists of a material adaptable to a living body. The shell preferably consists of a thermoresponsive polymer having a phase transition temperature in the vicinity of the body temperature of a target for drug administration. In order to achieve rapid drug efficacy, it is desirable that the shell susceptibly changes the characteristics of the shell membrane in the vicinity of the phase transition temperature. In the case where the shell is broken to release its inclusions at a temperature of the phase transition temperature or above, rapid release, as shown in FIG. 4, for example, is desired. In FIG. 4, a drug carrier containing magnetic fine particles is formed by containing magnetic fine particles 2 and a drug 3 in a shell 1. Furthermore, more preferably, the shell of a drug carrier containing magnetic fine particles consists of a thermosensitive liposome (vesicle).

Furthermore, in the preferred embodiment of the present invention, it is necessary to prevent non-specific adsorption in blood. Preferably, the shell of a drug carrier containing magnetic fine particles has an outermost shell membrane consisting of a lipid membrane such as liposome, and takes ionized form having the particle surface potential biased to either of the + side and the − side from the isoelectric point of blood.

In the meantime, in a preferred embodiment of the present invention aiming for providing gene therapy, the shell of the drug carrier has a phase transition temperature in the vicinity of the body temperature, and consists of a liposome modified by thermoresponsive polymers which become hydrophobic at a temperature of the phase transition temperature or above. The shell preferably has a double-coating structure of a membrane having a phase transition temperature of T1 or above and consisting of thermosensitive functional polymers which release a drug at a temperature of the phase transition temperature or above, inside of a liposome membrane modified by thermoresponsive polymers which become hydrophobic at a temperature of the phase transition temperature T1 or above.

In addition, a cooling effect of blood stream is known in intravascular heating. In a preferred embodiment of the present invention used for controlling timings of intravascular drug administration and of providing treatment for the purpose of increasing a drug concentration in the vicinity of a lesion site, the drug carrier is in a form of being coated for providing high resistance or being coated by a resin. Preferably, the shell of the drug carrier has a double structure in which the outside of a membrane consisting of thermoresponsive polymers having a phase transition temperature in the vicinity of the body temperature of a target of drug administration is further treated to have a coat offering high resistance to blood flow or coated by a resin.

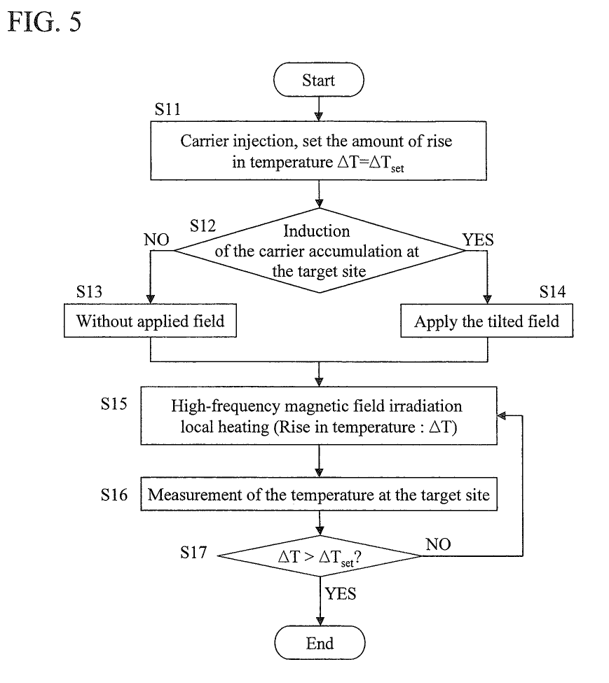

Next, an embodiment of therapy equipment using a drug carrier of the present invention will be described with reference to a flowchart in FIG. 5. Note that application of the present invention is not limited to the following concrete example.

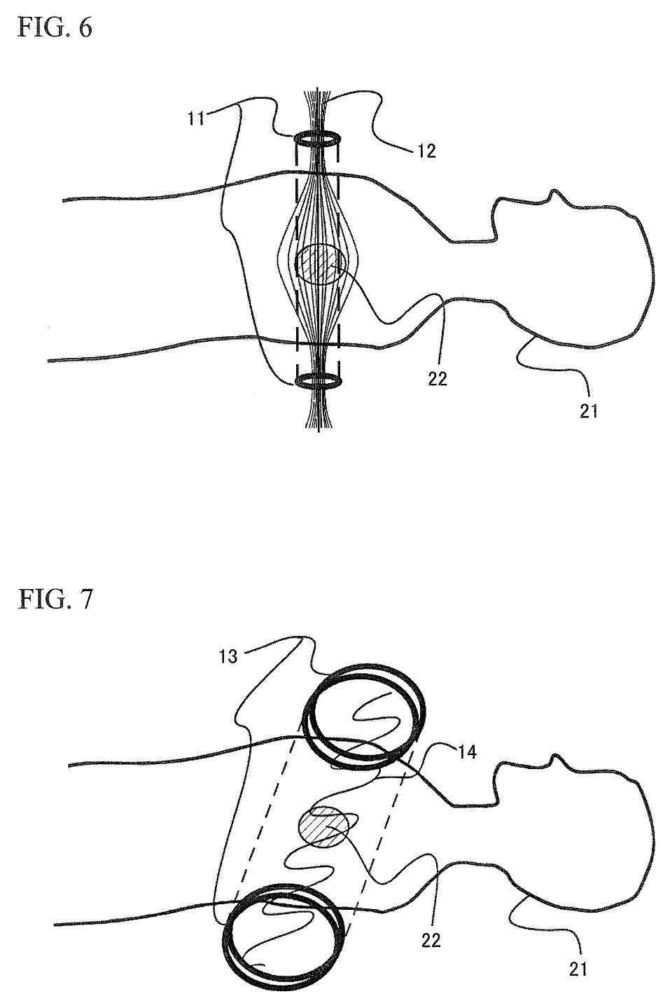

Firstly, an operator sets a value of rise in temperature ΔTₛₑₜ by intended heating in accordance with intended use, prescribes drug carriers suitable to the value of rise in temperature, and administers drug carriers (S11). An appropriate route of administration includes administrations inside of a target site, surrounding the target site, and inside intravascular. Preferred is a route of administration through the arterial or venous blood supply using a passive and active targeting method which is handled in a publicly-known DDS. Next, the drug carriers each containing magnetic fine particles are to be accumulated at a target site. Accumulation of the drug carriers is carried out by all publicly-known means in the present invention. A method of accumulation is determined according to intended use and the function of the drug carriers (S12). In the case of aiming for a temporary rise in drug concentration in the vicinity of the target site by rapid drug release during heating by use of a coating membrane having a high rate of change in the vicinity of the phase transition temperature, it is not particularly necessary to accumulate the drug carriers (S13). In the case where it is necessary to accumulate the drug carriers at a high concentration in an lesion tissue, a means is adopted in which drug carriers are highly-effectively accumulated at a target site by generating a static magnetic field gradient in the vicinity of the lesion tissue part, and further caused to stay at the position for a longer period of time by static magnetic field control (S14). For the generation of a high-gradient static magnetic field, as shown in a schematic view in FIG. 6, for example, a pair of coils 11 arranged across a target site 22 of a test body 21 is used. The pair of coils 11 provide a magnetic field gradient in which a generated static magnetic field component attenuates concentrically with the target site 22 at the center in a planar direction orthogonal to the direction of the magnetic field. As shown in the schematic view, magnetic flux lines 12 spatially spread outside of the coils 11. A magnetic field direction component attenuates inversely proportional to a cube of a distance from the target site 22 as a center in the planar orthogonal to the magnetic field direction.

Next, a high-frequency magnetic field is irradiated (S15). For generation of an alternating-current magnetic field used for a high-frequency magnetic field, as shown in FIG. 7, for example, a target site 22 may be arranged between a pair of coils 13 carrying an alternating current. An electromagnetic wave 14 used in the present invention is not particularly limited, but any electromagnetic wave can be used as long as it has a frequency capable of applying high-frequency dielectric heating to the magnetic fine particles, and a radiofrequency wave (a frequency in a range from 30 Hz to 300 MHz and a wavelength in a range from 1 m to 100 km) and a microwave (a frequency in a range from 300 MHz to 300 GHz and a wavelength in a range from 1 mm to 1 m) can be used. In addition, as for this electromagnetic wave, it is preferable to have a frequency of 100 MHz or less because it is poorly absorbed by water and thereby unlikely to non-specifically apply high-frequency heating to any substance other than magnetic fine particles. Timings for drug administration and treatment are controlled by monitoring the temperature of the target site (S16) and the state of high-frequency magnetic field irradiation is controlled with the high-frequency magnetic field being irradiated. It is judged whether or not the measured value of rise in temperature ΔT exceeds the target value of rise in temperature ΔTₛₑₜ (S17). If ΔT is below ΔTₛₑₜ, a high-frequency magnetic field is again irradiated (S15). When ΔT exceeds ΔTₛₑₜ, the treatment is terminated.

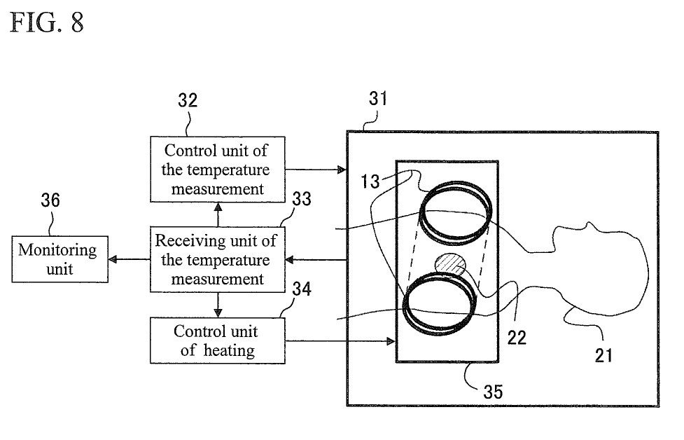

FIG. 8 is a schematic illustration of a configuration of a control unit for heating of therapy equipment of the present invention. This therapy equipment includes: a bed for holding a test body; a heating unit 35; a temperature measurement unit 31; a receiving unit of the temperature measurement 33; a control unit of the temperature measurement 32; a control unit of heating 34; and a monitoring unit 36. The heating unit 35 includes a coil 13 for high-frequency magnetic field generation. Additionally, the heating unit 35 may include a coil for static magnetic gradient generation as shown in FIG. 6. A value of rise in temperature ΔT after irradiation of a magnetic field from the temperature measurement unit 31 is received by the receiving unit of the temperature measurement 33, and temperature distribution in the vicinity of the target site is monitored by the monitoring unit 36. If the value of rise in temperature ΔT is below a target value of rise in temperature ΔTₛₑₜ set in advance as a target, the heating unit 35 irradiates a high-frequency magnetic field in accordance with a signal from the control unit of heating 34. In addition, the control unit of the temperature measurement 32 causes the temperature measurement unit 31 to measure a value of rise in temperature. When ΔT exceeds ΔTₛₑₜ, the high-frequency magnetic field irradiation by the heating unit 35 is brought to a halt, and the treatment is terminated. For the control unit, PC can be used.

For the temperature measurement unit 31, known temperature measurement using an MRI system is applicable. Alternatively, the temperature measurement may be carried out by methods, such as imaging of a heating site by use of an infrared camera and imaging of a heating site by placing, in the vicinity of a target site, an apparatus formed by arranging infrared imaging sensors, as described in Japanese Patent Application Publication No. 2007-057449, in a matrix formation. The temperature measurement unit 31 is preferably composed of an MRI system capable of stably calculating temperature changes in time series inside of a test body even if the test body is moving because the MRI system performs time-series multi-echo imaging for obtaining multiple MR images having different echo times at the same nuclear magnetic excitation timing, and calculates three-dimensional or two-dimensional temperature distribution of the test body in each time phase by performing signal processing of the images.

Hereinafter, a drug carrier of the present invention will be concretely described. It should be noted that the present invention is not limited to the following Examples. Hereinafter, the specific gravities of a drug and surrounding cells are calculated roughly at 1.

### FIRST EXAMPLE

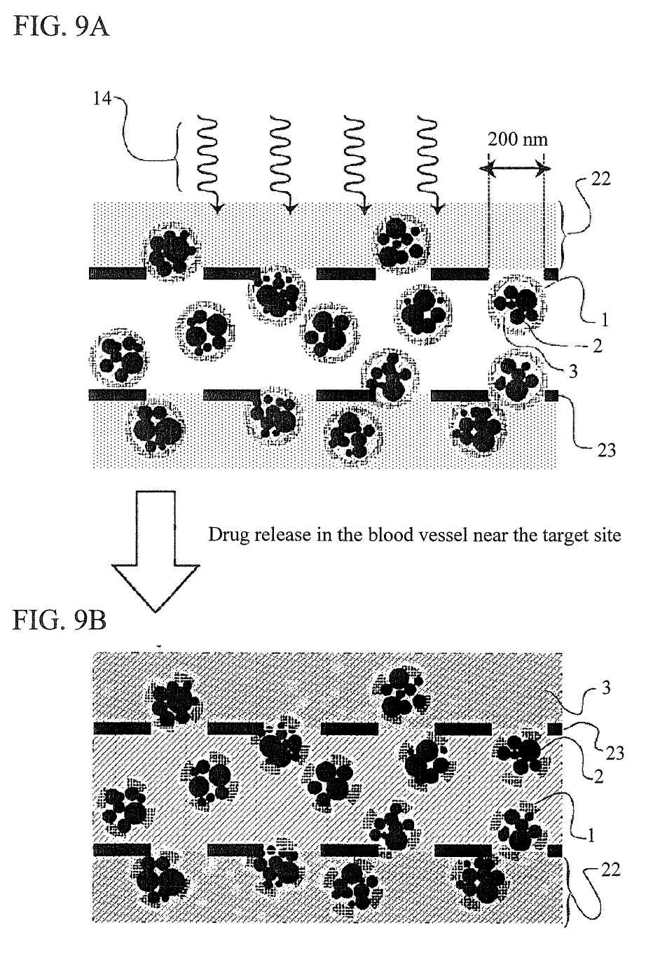

As drug carriers containing magnetic fine particles, a publicly-known liposome having a transition temperature of 39° C., being modified with thermoresponsive polymers 1, and having a size of 200 nm. For example, N-isopropylacrylamide copolymers (K. Yoshino, A. Kadowaki, T. Takagishi, K. Kono, Bioconjugate Chemistry, 15, 1102-1109, 2004) were used. As shown in FIG. 9, a drug 3 and single-magnetic domain nickel fine particles 2 having an anisotropic magnetic field Hₖ of 40 Oe and a saturated magnetization of 510 emu/cm² were inserted into a vesicle modified with thermoresponsive polymers 1. The magnetic fine particles used here had an average particle diameters of 20 nm and a standard deviation σ of particle diameter distribution of 10 nm (σ=0.5d). In this case, H_{c}/Hₖ=1.4. Accordingly, 3Hₖ/Mₛμ₀≈0.0195<Φ, when the volume fraction Φ=0.1.

The drug was injected in a route of administration through the venous blood supply, and a target site 22 was irradiated with a high-frequency magnetic field 14 having a frequency of 200 kHz at a magnetic-field intensity of 1000 Oe a few minutes later. At a rough estimate using the specific heat of the drug carriers and the specific heat of water, which is 4.2×10⁹ Jg⁻³K⁻¹, the period of irradiation is approximately 200 seconds to achieve a rise in temperature of 3° C. of the drug carriers in the vicinity of the target site. If the body temperature is assumed to be 36° C., the temperature of the drug carriers rises to 40° C. by irradiation of a high-frequency magnetic field for approximately 4.5 minutes. As a result, from the state before the irradiation illustrated in FIG. 9A, vesicles 1, serving as drug carriers, staying in the vicinity of the target site are deformed, and the drug 3 is released. The drug 3 then permeates vessel walls 23 as shown in FIG. 9B, and reaches the target site 22. Hence, during the treatment immediately after the drug carriers are injected to the blood, the drug concentration can be increased due to locally-induced heating by a high-frequency magnetic field.

### SECOND EXAMPLE

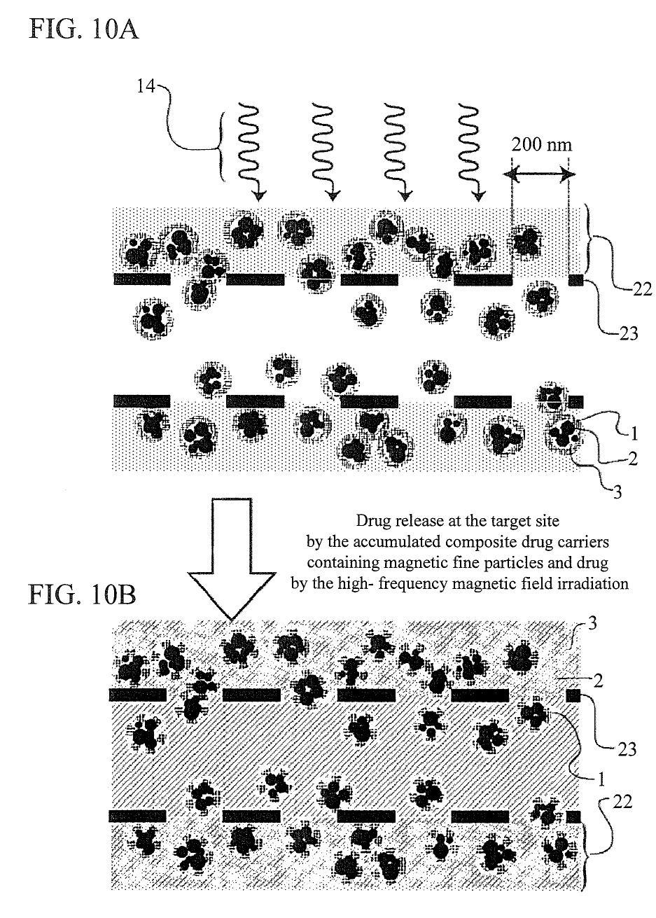

Using a thermoresponsive polymer micelle, poly(IPAAm-co-DMAAm)-block-poly(DL-lactide), having a transition temperature of 40° C., described in Supramolecular Design for Biological Applications (2002), chapter 11, Editor(s): Yui, Nobuhiko, Publisher: CRC press LLC, Boca Raton, Fla. as a shell 1 containing a drug and magnetic fine particles, drug carriers containing magnetic fine particles were produced. As shown in FIG. 10, a drug carrier having an average particle diameter of 100 nm contained a drug 3 and FePt particles 2 having an anisotropic magnetic field Hₖ of 1000 Oe, a saturated magnetization of 1140 emu/cm², an average particle diameter of 10 nm, and a standard deviation of 8 nm. In this case, H_{c}/Hₖ=2.1. Accordingly, 3Hₖ/Mₛμ₀≈0.21<Φ, when the volume fraction Φ=0.3.

The drug was injected in a route of administration through the venous blood supply, and a target site 22 was irradiated with a high-frequency magnetic field 14 having a frequency of 200 kHz at a magnetic-field intensity of 1000 Oe one day later. Accumulation of drug carriers due to the EPR effect (Enhanced Permeability and Retention: the effect that DDS drugs tend to accumulate in cancer tissues because neovascular walls of cancer tissues have a high degree of leaking from normal vessel walls to cancer cell tissues.) is described as follows, for example. According to Supramolecular Design for Biological Applications (2002), Chapter 11, Editor(s): Yui, Nobuhiko, Publisher: CRC press LLC, Boca Raton, Fla., it is reported that approximately 10% of a total amount of injected drug carriers accumulates per 1 g of tumor 24 hours after the drug injection when 10 mg of a drug per 1 kg of body weight is injected in a study using a mouse tumor as a target site and drug carriers modified with polymer micelles. If the body weight is assumed to be 0.05 kg, 0.05 mg of the drug accumulates per 1 g of tumor. In the present Example, the particle diameter of the carriers is ½ of that in First Example. Accordingly, it is assumed that the vessel permeability is higher, and that, one day after the drug injection, as shown in FIG. 10A, the carriers permeated through vessel walls 23 reach and stay in a tumor tissue at a higher concentration than that in FIG. 9. In this case, if a volume of drug per one carrier is 20%, the density of drug carriers having a size of 100 nm is one per 4 μm³ of a tumor site. Furthermore, when the accumulation is doubled by generating a static magnetic field gradient, an anisotropic magnetic field of FePt particles is 1000 Oe, and the specific heat of the drug carrier and surrounding cells is 4.2×10⁹ Jg⁻³K⁻¹, the period of irradiation required for a rise in temperature of 9° C. of the drug carriers in the vicinity of the target site is approximately 16 minutes. In a case where the body temperature is assumed at 36° C., the temperature of the tumor site rises to 45° C. after the irradiation of high-frequency magnetic field for approximately 16 minutes. As a result, as shown in FIG. 10B, drug release by deformation of drug carriers staying in the target site and drug release also from drug carriers located in the vicinity of the target site are promoted. Accordingly, as the drug reaches the target site 22 permeating through vessel walls 23, hyperthermia therapy can be accomplished at the target site 22.

Unlike full-body heating for an extended period of time at a maximum temperature of 43° C. by irradiation using an rf wave of 8 MHz for more than 30 minutes in a conventional high-frequency dielectric heating method, it is possible to apply heating at a temperature of 43° C. or above limited to a local site for a short period of time. Moreover, magnetic fine particles released to a tumor site naturally form clusters with aggregation. Accordingly, in the case where a condensed structure has a volume fraction satisfying the formula (5), the heating efficiency rises dependently on the size distribution of the clusters according to the curve shown in FIG. 3. Therefore, it is possible to achieve efficient simultaneous progress of local hyperthermia and chemotherapy.

### THIRD EXAMPLE

As drug carriers containing magnetic fine particles, a hybrid-type cationic liposome 1 containing a drug 3 and magnetic fine particles 2 was used. The hybrid-type cationic liposome 1 consists of a phospholipid modified with thermoresponsive polymers (for example, NIPMAM-NIPMAM copolymer) having a transition temperature of 40° C., which were synthesized according to K. Kono, R. Nakai, K. Morimoto, and T. Takagishi, FEBS Lett., 456, 306-310 (1999), and of a micelle surfactant.

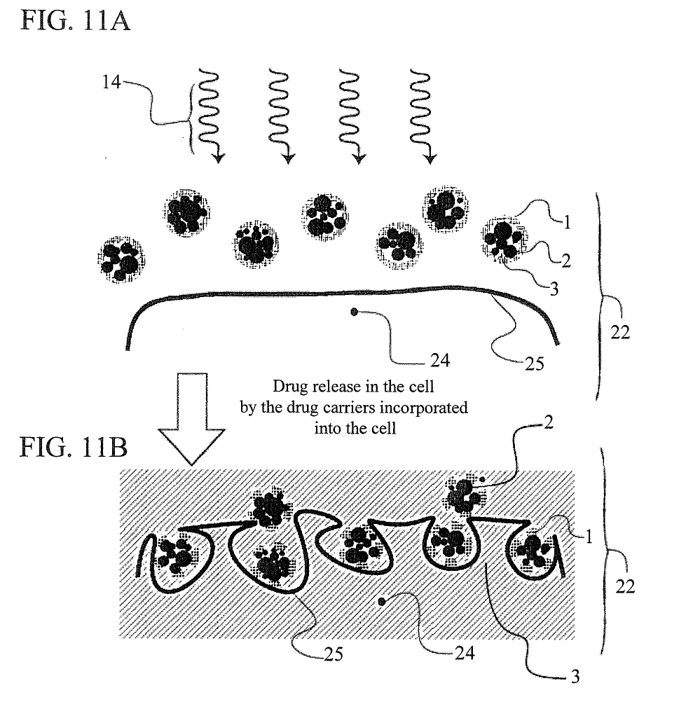

As shown in FIG. 11, drug carriers having an average size of 100 nm contained single magnetic-domain iron particles having an anisotropic magnetic field Hₖ of 400 Oe, a saturated magnetization of 1710 emu/cm², an average particle diameter of 10 nm, and a standard deviation of 5 nm. In this case, H_{c}/Hₖ=1.4. Accordingly, 3Hₖ/Mₛμ₀≈0.06<Φ, if the volume fraction Φ=0.2.

The drug was injected in a route of administration through the venous blood supply, and a target site 22 was irradiated with a high-frequency magnetic field 14 having a frequency of 100 kHz at a magnetic-field intensity of 400 Oe one day later. As in Second Example, the size of the carriers in the present Example is relatively small. Accordingly, it is assumed that the vessel permeability is higher, and that, one day after the drug injection, as shown in FIG. 11A, the carriers permeated through vessel walls 23 reach and stay in the vicinity of a target site tissue at a high concentration. It is known that a cationic liposome, as shown in FIG. 11B, changes its liposome characteristics at a temperature of the transition temperature or above, and thereby is promoted to be incorporated into a cell. When the specific heat of the drug carriers is 4.2×10⁹ Jg⁻³K⁻¹, the period of irradiation required for a rise in temperature of 6° C. only in the drug carriers is approximately 40 seconds. If the body temperature is assumed to be 36° C., the liposome characteristics on the surface of the drug carriers change due to irradiation of a high-frequency magnetic field for approximately 40 seconds, and the drug carriers having reached to the target site 22 permeating through the vessel walls 23 as shown in FIG. 11A invade a cell 24, and release the drug. As a result, in the target site 22, it is possible to incorporate drug carriers into cells and to perform drug release inside the cells. Hence, more rapid drug efficacy can be expected.

The present invention utilizes the fact that it is possible to increase heating efficiency of magnetic fine particles in a condensed system by 2 to 4 times by controlling the particle diameter distribution compared to the case where particle diameter distribution is not controlled. The site-oriented high-frequency dielectric heating according to the present invention can be used for various applications, such as double targeting in DDS which is a method for delivering drugs, heating control in hyperthermia and the like. Moreover, magnetic fine particles are aggregated inside of a drug carrier in the present invention. Accordingly, while preventing the magnetic fine particles from dispersing all over inside a living body, the present invention can constantly keep enough magnetic fine particles required for effective high-frequency heating together in a group.

### EXPLANATION OF REFERENCE NUMERALS

1 shell
2 magnetic fine particle
3 drug
11 coil for static magnetic gradient generation
13 coil for high-frequency magnetic field generation
14 high-frequency magnetic field
21 test body
22 target site
23 vessel wall
24 cell in the target tissue
25 cell wall in the target tissue
31 temperature measurement unit
32 control unit for the temperature measurement
33 receiving unit of the temperature measurement
34 control unit of heating
35 heating unit
36 monitoring unit

What is claimed is:

1. A drug carrier comprising:
a drug;
a plurality of magnetic fine particles being aggregated; and
a shell containing the drug and the plurality of magnetic fine particles, wherein
the plurality of magnetic fine particles are single magnetic-domain magnetic fine particles, and have a standard deviation σ satisfying 0.8d>σ>0.4d where d denotes an average particle diameter, and
the shell has an outer diameter in a range from 10 nm to 200 nm.

2. The drug carrier according to claim 1, wherein the drug carrier includes a carrier in which a standard deviation σᵢ of particle diameters of magnetic fine particles in each carrier i satisfies 0.8dᵢ>σᵢ>0.4dᵢ where dᵢ denotes an average particle diameter of an assembly of magnetic fine particles contained in each carrier.

3. The drug carrier according to claim 1, wherein the magnetic fine particles are made of any one of iron, cobalt and nickel, or any one of an alloy, an oxide and a nitride of iron, cobalt or nickel.

4. The drug carrier according to claim 1, wherein the magnetic fine particles have a coercivity H_{c} in an aggregated powder compacting state of fine particles in a range from approximately one to five times an anisotropic magnetic field Hₖ.

5. The drug carrier according to claim 1, wherein a volume fraction Φ₀ of the magnetic fine particles, a saturated magnetization Mₛ, and an anisotropic magnetic field Hₖ satisfy the following relationship:
$${φ}_{0} > \frac{3 {H}_{k}}{{M}_{s} {μ}_{0}}$$

6. The drug carrier according to claim 1, wherein the shell is composed of a thermoresponsive polymer having a phase transition temperature close to a body temperature of a target of drug administration.

7. The drug carrier according to claim 6, wherein the shell is a vesicle modified with a thermosensitive liposome.

8. The drug carrier according to claim 6, wherein the shell is a thermosensitive micelle.

9. Therapy equipment, comprising:
a holding table for holding a test body administered drug carriers each including a drug, a plurality of magnetic fine particles being aggregated, and a shell containing the drug and the plurality of magnetic fine particles, the plurality of magnetic fine particles being single magnetic-domain magnetic fine particles and having a standard deviation σ satisfying 0.8d>σ>0.4d where d denotes an average particle diameter, and the shell having an outer diameter in a range from 10 nm to 200 nm;
a high-frequency magnetic field irradiation unit for applying high-frequency dielectric heating to the drug carriers aggregated at a target site of the test body;
a temperature monitor for monitoring the temperature of the target site; and
a control unit for causing the high-frequency magnetic field irradiation unit to operate until a rise in the temperature monitored by the temperature monitor reaches a predetermined target value of rise in temperature and for stopping the high-frequency magnetic field irradiation unit from operating when the temperature rise reaches the target in temperature rise value.

10. The therapy equipment according to claim 9, further comprising a means for generating a gradient magnetic field for aggregating the drug carriers at the target site of the test body.

11. The therapy equipment according to claim 9, wherein a temperature monitoring function by nuclear magnetic resonance imaging utilizing a proton nuclear magnetic resonance frequency proportionally related to a temperature is used as the temperature monitor.
